(19) United States
(12) Patent Application Publication
(10) Pub. No.: US 2008/0188864 A1
(43) Pub. Date: Aug. 7, 2008

(54) LITHOTRIPSY BASKET DRILL

(75) Inventor: RICHARD W. DUCHARME, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 11/969,056

(22) Filed: Jan. 3, 2008

Related U.S. Application Data
(60) Provisional application No. 60/880,222, filed on Jan. 12, 2007.

(51) Int. Cl. A61B 17/22; A61B 17/00

(52) U.S. Cl. 606/127; 606/128; 606/80

(57) ABSTRACT

A lithotriptor device embodiment is provided including a lithotriptor that has a proximal handle, an elongate shaft extending distally from the handle with a lumen extending through a major length of the elongate shaft, and a wire basket distally attached to a drive wire. The drive wire extends through the lumen of the elongate shaft and is operatively connected to the handle. The device includes a drill mechanism assembly with a drill bit disposed near the distal end of the elongate shaft.

### CROSS-REFERENCE TO RELATED APPLICATIONS

This application claims priority to U.S. Provisional Application Ser. No. 60/880,222, filed Jan. 12, 2007, which is incorporated herein by reference in its entirety.

### FIELD OF THE INVENTION

The present invention relates generally to medical devices, and relates more specifically to devices and methods for mechanical lithotripsy of stones (calculi) such as bile stones.

### BACKGROUND

The gall bladder is an organ that stores bile secreted by the liver. The cystic duct from the gall bladder merges with the common hepatic duct, forming the common bile duct. A number of medical conditions are associated with various disorders, diseases, and injuries associated with the bile duct.

Choledocholithiasis is a medical condition associated with the entry of a biliary calculus (bile stone) into the bile duct. Obstruction of the bile duct can be excruciatingly painful for a patient suffering therefrom, and can cause nausea, fever, vomiting, and jaundice. Complete, persistent obstruction of the common bile duct can cause cholangitis, a life threatening infection of the biliary tree, which is a medical emergency. An obstruction of the common bile duct can also lead to an obstruction of the pancreatic duct, which may cause pancreatitis.

Several methods of treatment are used to remove the gall bladder and stones, including open surgery or laparoscopic surgery. Less invasive treatments may be used as well. For example, the stones may be removed endoscopically using, for example an endoscopic retrograde cholangiopancreatography (ERCP) procedure, without having to create any external incisions. In this minimally invasive surgical technique, an endoscope is directed through the patient's esophagus to a location adjacent the Sphincter of Oddi, where the bile duct opens into the duodenum. Typically, a sphincterotome is used to cannulate and widen the sphincter opening to ease access into the bile duct for stone retrieval. A device including a basket deployable from a lumen of a catheter may then be directed into the bile duct to capture stones for removal.

In some instances the stones are too large to pass through even a widened Sphincter of Oddi. If more invasive surgical techniques are to be avoided, then the stone must be crushed or broken into smaller pieces for removal (lithotripsy). A number of devices are known in the art for breaking up the stones. One such device is a mechanical lithotriptor basket device 100 comprising a wire basket 104 mounted on the distal end of an elongate basket wire 102, which is guided through a catheter 110 to a location such that the basket 104 can be directed around a stone 106 (See FIGS. 1A-1C). Once the basket 104 is around the stone 106, the basket 104 is retracted toward and into the catheter 110, such that its internal volume is reduced. The compressive force caused thereby crushes/breaks the stone 106 into smaller pieces (See FIG. 1D) so that it can be removed or allowed to pass.

In some circumstances, the retraction and compaction of the basket 104 may be accomplished by a user directly pulling the basket wire 102 proximally (e.g., with a standard handle such as a three-ring handle or a flanged-spool/stem handle). However, because some stones may be resistant, it is often necessary to provide mechanical advantage to aid in crushing of the stone 106. A number of devices have been used to address this need by introducing increased force/greater mechanical advantage from a proximal portion of a lithotripsy device assembly. One device that has been used for this purpose is a reel-type device embodied in the Soehendra® Mechanical Lithotriptor (Cook Endoscopy). FIG. 2A illustrates a reel-type lithotriptor accessory handle 220 and FIGS. 2B-2E depict a method of use. FIG. 2B shows the distal portion of a lithotripsy device 200 including a lithotripsy basket 202 at the distal end of a basket wire 204 and catheter 210 fully engaged with a stone 206. FIGS. 2C-2D depict how the proximal end of the basket wire 204 and catheter 210 are mounted to the lithotriptor accessory handle 220 after removal of an initial proximal structure (e.g., a three-ring handle). FIG. 2E shows how the lithotriptor accessory handle 220 is actuated to crush the stone 206. Other presently-available devices for providing mechanical advantage when a stone is resistant to crushing also require the use of additional accessory tools that must be assembled to the lithotripsy device 200 to provide mechanical advantage. This requirement of extra steps and extra hardware reduce the efficiency that is most desirable during surgical procedures. Thus, there is a need for a lithotripsy device that provides other means for disrupting a recalcitrant stone requiring extra steps and devices.

### BRIEF SUMMARY

In one aspect, embodiments of the present invention may provide a lithotriptor device including a proximal handle; an elongate shaft extending distally from the handle with a lumen extending through a major length of the elongate shaft; a wire basket distally attached to a drive wire, the drive wire extending through the lumen of the elongate shaft and operatively connected to the handle; and a drill mechanism assembly comprising a drill bit and disposed near the distal end of the elongate shaft.

In another aspect, embodiments of the present invention may provide a method for crushing an object including the steps of providing a medical lithotriptor device comprising a proximal handle, an elongate shaft extending distally from the handle with a lumen extending through a major length of the elongate shaft, a basket distally attached to a drive wire, the drive wire extending through the lumen of the elongate shaft and operatively connected to the handle, and a drill mechanism assembly comprising a drill bit and disposed near the distal end of the elongate shaft; then, engaging the basket around an object and actuating the handle such that the drive wire is drawn proximally into the elongate shaft and the basket is drawn tightly around the object.

In yet another aspect, embodiments of the present invention may provide a lithotriptor device including a proximal handle, an elongate shaft extending distally from the handle with a lumen extending through a major length of the elongate shaft, a wire basket distally attached to a drive wire, the drive wire extending through the lumen of the elongate shaft and operatively connected to the handle, and a fluid turbine-driven drill means comprising a drill bit, said drill means being disposed such that the drill bit project distally from the elongate shaft.

### BRIEF DESCRIPTION OF THE DRAWINGS

FIGS. 1A-1D depict the function of a lithotriptor basket;

FIG. 2A illustrates a prior art lithotriptor handle accessory for increasing mechanical advantage;

FIG. 2B shows a lithotriptor basket engaging a biliary calculus;

FIGS. 2C-2E depict a method of using the prior art lithotriptor handle accessory with a lithotripsy device;

FIG. 3 illustrates a first embodiment of a lithotriptor device including a drill component;

FIG. 4 shows a second embodiment of a lithotriptor device including a drill component;

FIGS. 5-5A depict sectional and end views of a lithotriptor device embodiment including a drill component;

FIGS. 6-6A depict sectional and end views of a lithotriptor device embodiment including a drill component; and

FIGS. 7A-7E illustrate a method of using a lithotriptor device embodiment including a drill component.

### DETAILED DESCRIPTION

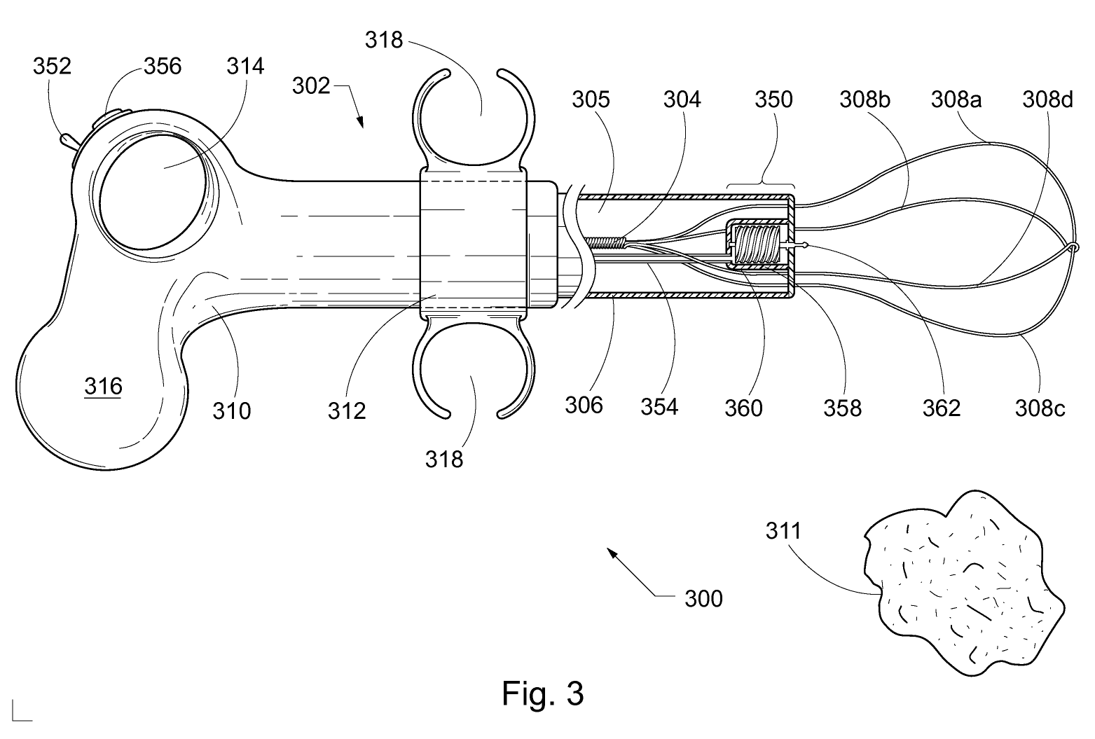

A first embodiment of a drill-equipped lithotriptor 300 is illustrated in FIG. 3, with the distal portion being shown diagrammatically (not to scale) in a partially sectioned view. In addition to a handle 302, the lithotriptor 300 includes a drive wire 304, circumscribed by and axially slidable within a lumen 305 of an elongate shaft embodied as an outer sheath 306 that extends distally from the handle 302. The drive wire 304 may include a single structure that is attached to the basket wires 308a-308d, it may include a proximal portion of the basket wires 308a-308d braided or otherwise held together or extending independently, or it may include another drive wire structure appropriate for use with a lithotriptor.

In the illustrated embodiment, the distal end of the drive wire 304 includes a lithotripsy basket 308 formed of basket wires 308a-308d, which is shown in FIG. 3 as being disposed adjacent a biliary stone 311. The handle 302 includes a modified three-ring handle design. The stem (thumb-ring) portion 310 is attached to the proximal end 305 of the outer sheath 306. The spool (finger-ring) portion 312 is attached to the drive wire 304 such that axial movement of the spool 312 relative to the stem 310 causes corresponding axial movement of the drive wire 304 within the outer sheath 306 (the “spool” is known as such due to its general resemblance in longitudinal cross-section to a spool of the type used for thread, cable, etc). In preferred embodiments, the handle will be constructed of materials known in the art to be durable and suited for multiple sterilizations such as metals, resins, composites, or combinations thereof. For a disposable handle, certain injection-molded polymers may be appropriate. In preferred embodiments, load-bearing pivot points/axes (e.g., pivot pins) will be made of steel or a similarly rigid and durable material. (NOTE: FIGS. 3-7E are not drawn to scale; those of skill in the art will appreciate that the components may be differently proportioned and more compactly arranged than is depicted in these diagrammatic illustrations).

The proximal portion of the stem 310 includes a thumb ring aperture 314. An optional broad body 316 surrounding the aperture 314 preferably is shaped to fit comfortably in a user's palm during an operation when the spool 312 is pulled along the stem 310 toward the proximal end. The spool 312 includes two finger ring apertures 318. Thus, the handle 302 includes structure that allows a user comfortably to draw the spool 312 distally along the stem 310 by engaging her fingers into the finger ring apertures 318 and either engaging his/her thumb into the thumb ring aperture 314 or placing the broad proximal body 316 against his/her palm.

The handle 302 also includes an actuation switch 352 for actuating a distal drill mechanism 350 that is described below. Alternatively, the actuation switch may be mounted to a structure other than the handle such as, for example, a device configured to provide pressurized fluid for operating the distal drill mechanism. As is known in the art, the switch 352 may be configured as a dual-state (on/off) switch or as a rheostat switch allowing continuous and/or graduated/incremental control of the drill (e.g., speed of rotation), and the switch may be located separate/distant from the handle 302 (e.g., as a foot-actuated switch).

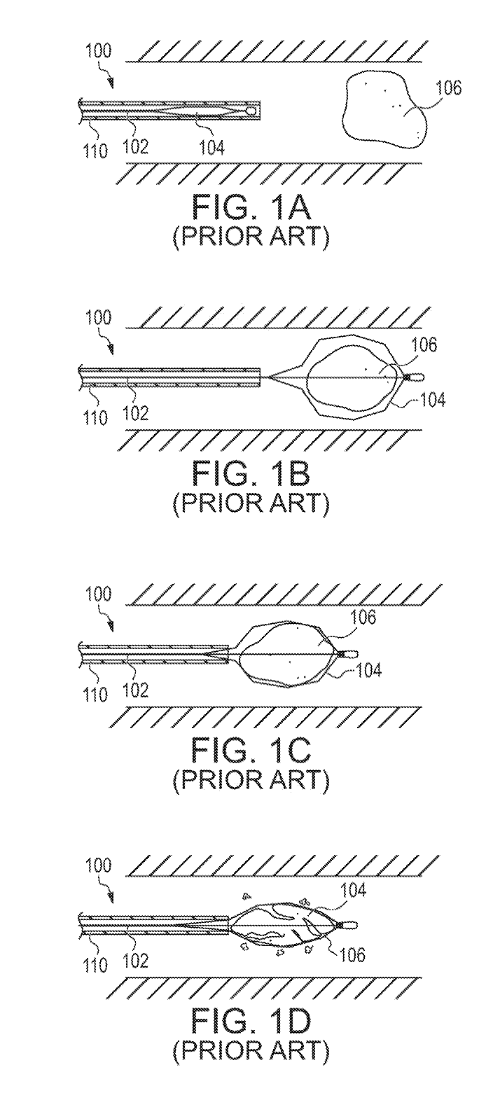

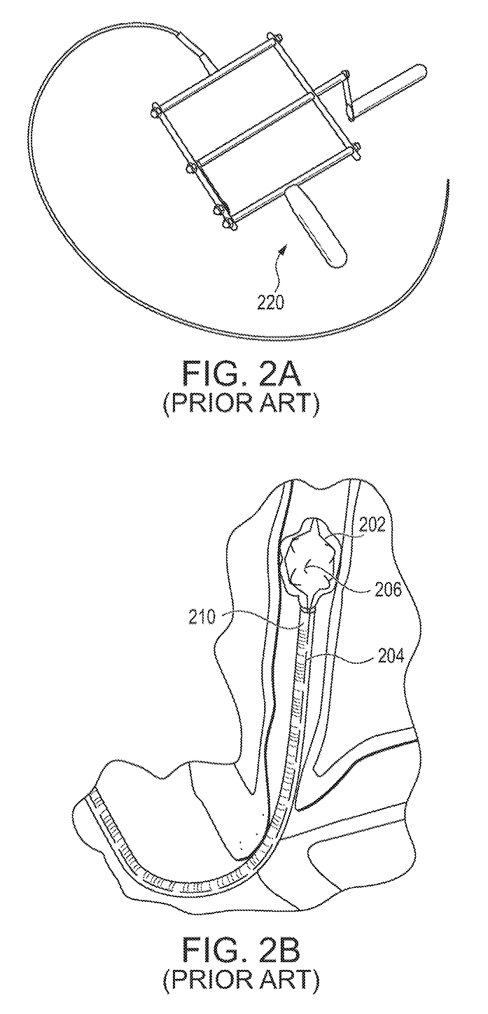

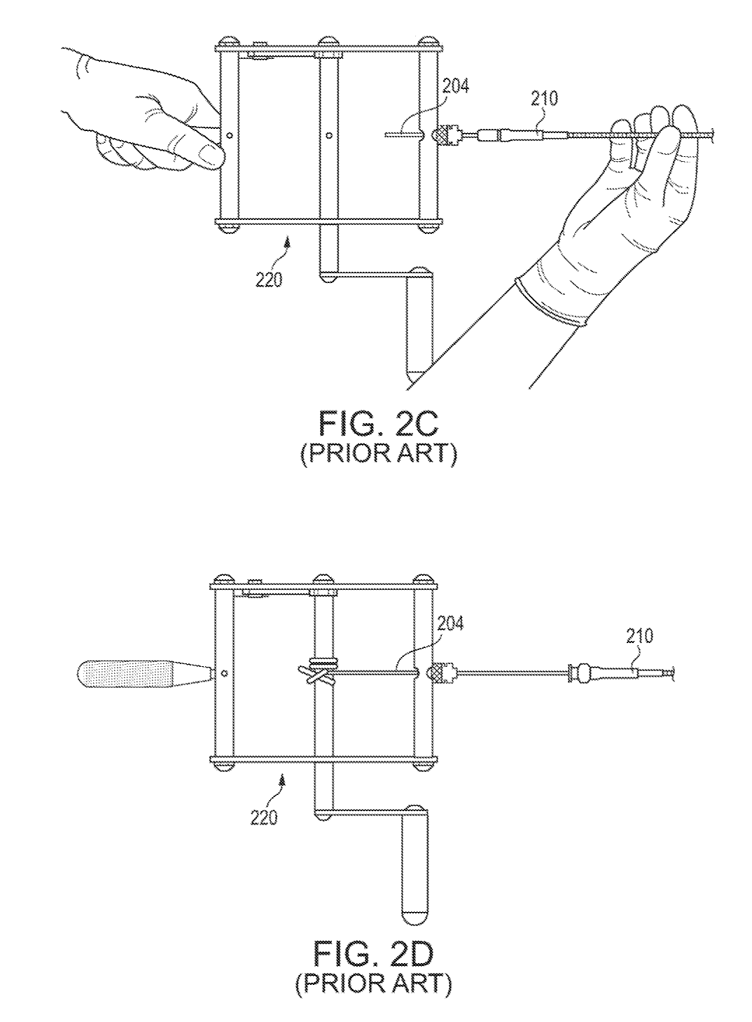

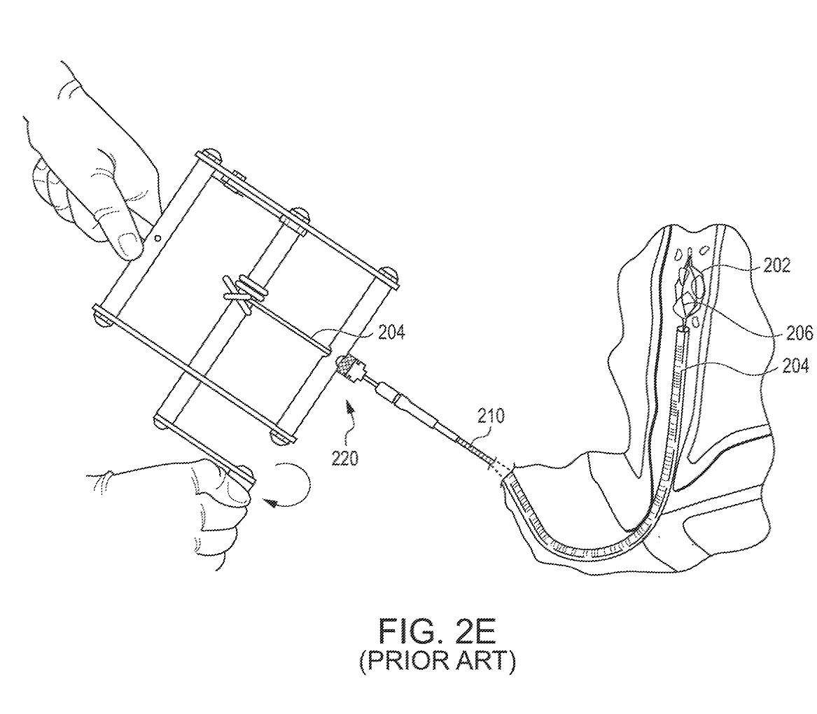

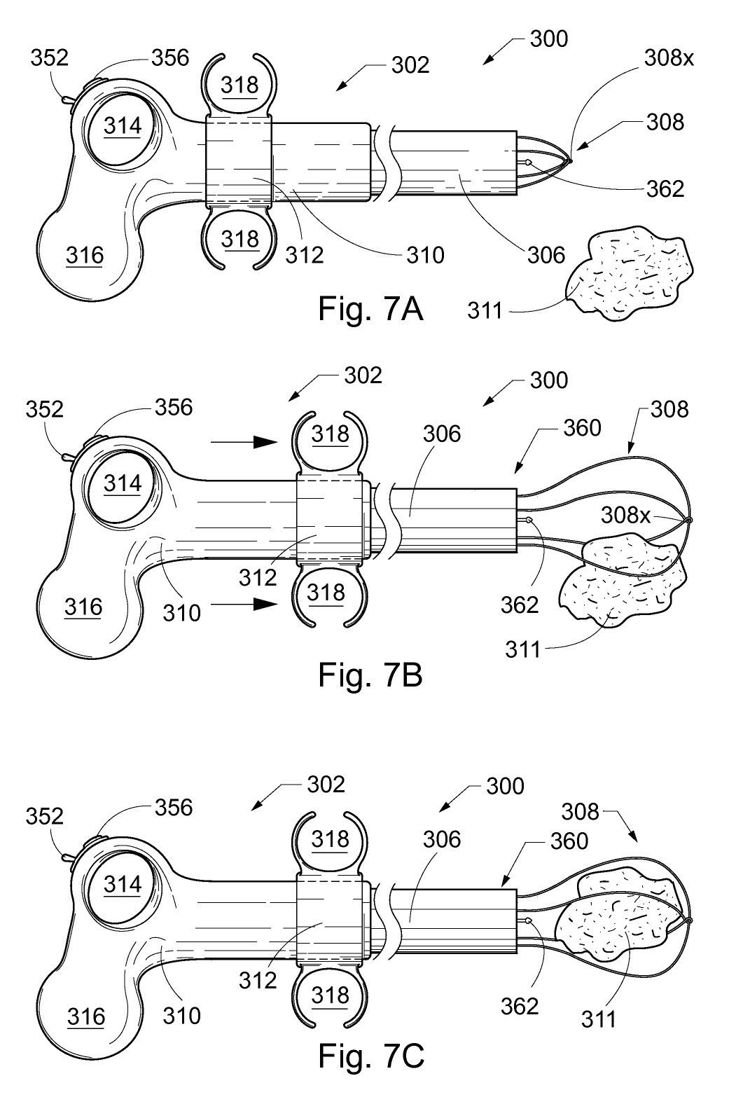

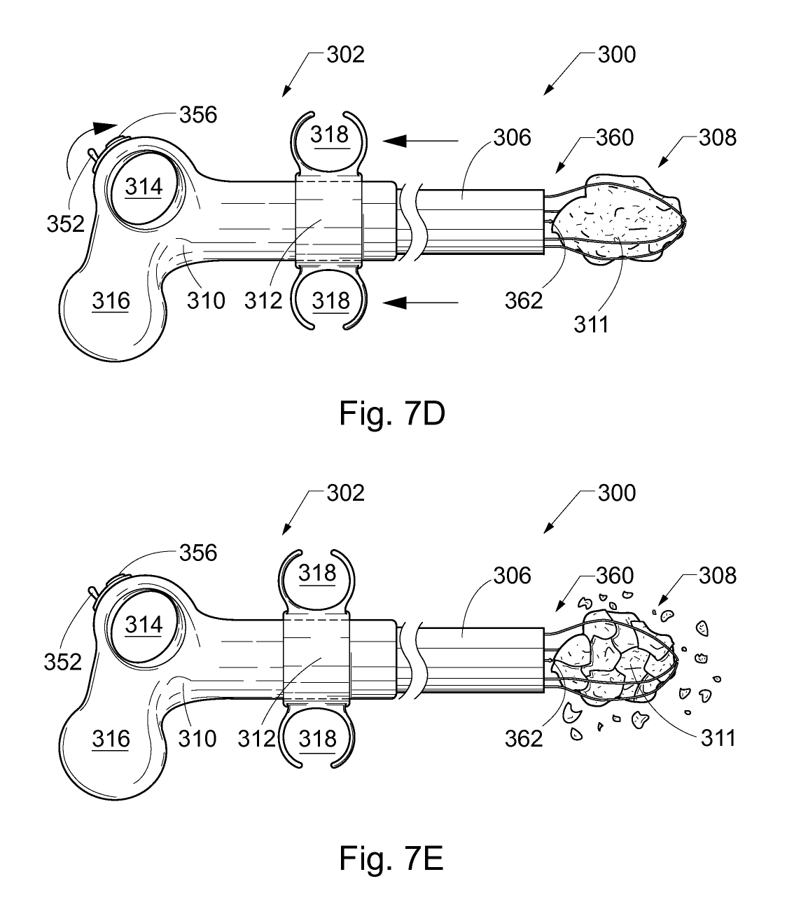

As is illustrated and discussed below with reference to FIGS. 7A-7E, the handle 302 may be actuated in the same fashion as a standard three-ring handle by pulling the spool 312 proximally along the stem 310 and toward the broad proximal body 316 using the finger ring apertures 318. The outer sheath 306 extending distally from the handle 302 preferably is a metal sheath such as a metal coil or cabled metal sheath of the type in the aforementioned Soehendra® Mechanical Lithotriptor (Cook Endoscopy #G21604 & G21860). The sheath structure preferably provides sufficient longitudinal strength to maintain integrity during a lithotripsy operation and preferably provides sufficient distal radial strength/integrity to resist expansion when the basket 308 is drawn therein to exert compressive force on the stone 311 (in a manner similar to that described with reference to FIGS. 1A-1D).

The present embodiment of the lithotriptor 300 includes a drill mechanism 350. The drill mechanism 350 is constructed in a manner similar to a dental drill (also called a dental handpiece). Specifically, a contra-angle fluid turbine, electric motor, or other means known in the dental drill art (and in medical arts using similar devices on, for example, bone) is used to rotate a drill bit 362 as described below. The drill bit 362—also known in the art as a “burr”—preferably includes an abrasive distal end portion such as a diamond dust-coated semispherical surface, and may range in length from about 1 mm to 10 mm or more. Examples of drill assemblies that include aspects appropriate for adapted use in embodiments disclosed herein include those described, for example, in U.S. Pat. Nos. RE30,356; 3,906,635; 4,470,813; and 4,786,251. Those of skill in the art will appreciate that many different drill embodiments are known in the art and are readily adaptable for use within the scope of the present invention. As is shown in FIG. 7A, the basket 308 may include a rounded tip 308x to present an atraumatic distal end and to protect the drill bit 362.

A first embodiment of the drill mechanism 350, shown in the detail sectional view of the distal lithotriptor section in FIG. 3, includes a fluid line 354 extending from the handle 302 to the drill head 360 adjacent the distal end of the outer sheath 306. The fluid line 354 provides a path of fluid communication from an entry port 356 of the handle 306 to a turbine drive mechanism in the drill head 360. As is well-known in the relevant drill art, a flow of pressurized fluid (e.g., air, an aqueous or non-aqueous solution) through the fluid line 354 and the turbine drive mechanism activates/rotates a turbine 358 of the turbine drive mechanism, which—in turn—rotates the drill bit 362. In certain embodiments of the present invention, the drill bit 362 and the means driving it may be configured to move the bit in a reciprocal/oscillating semi-rotating fashion (wherein, for example, the drill bit rotates clockwise a first predetermined number of degrees, then counterclockwise a second predetermined number of degrees, and repeats).

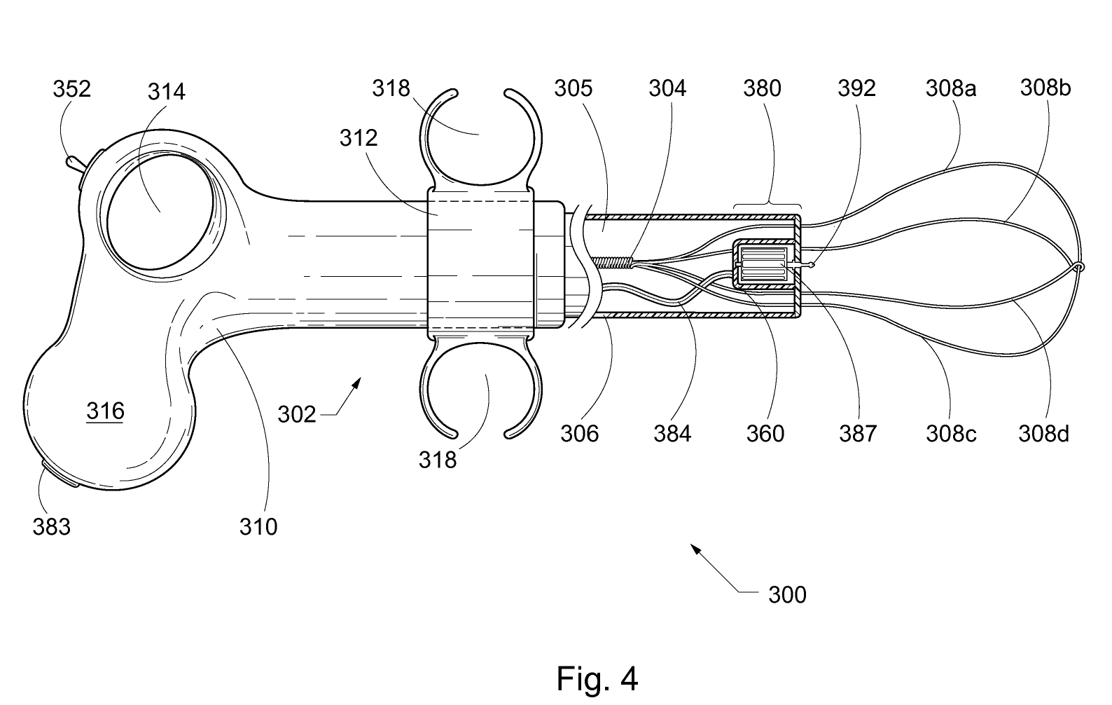

A second embodiment of the lithotriptor device 300 may be equipped with an electrically-driven drill mechanism 380, shown in FIG. 4 (and using the same handle configuration as the embodiment of FIG. 3) includes an electrical communication line 384 extending from the handle 302 to the drill head 360 adjacent the distal end of the outer sheath 306. The electrical communication line 384 provides a path for an actuation signal from an actuation switch 382 and electrode connection 383 of the handle 306 to an electronic drive mechanism 387 in the drill head 360. As is well-known in the relevant drill art, an electronic motor can be used to operate the drill bit 392, such as, for example, by using an electronically-driven rotor to spin the drill bit 392.

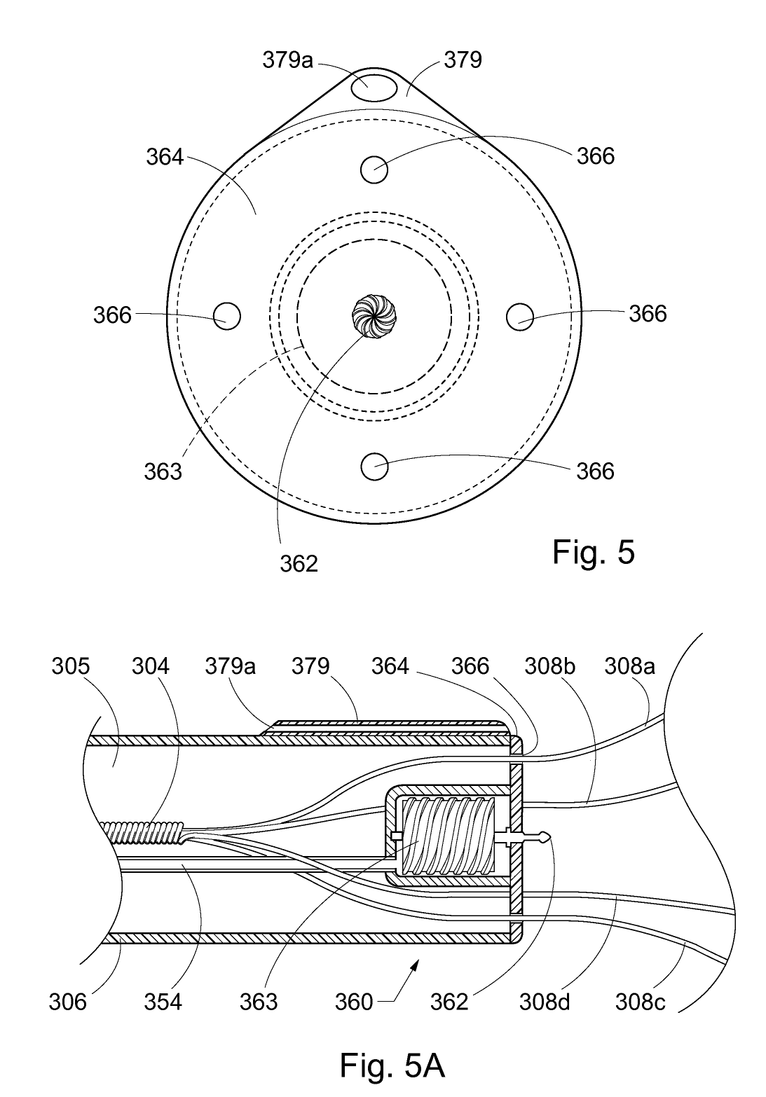

A first embodiment of the drill head 360 is shown from a detailed end view in FIG. 5. The drill head 360 includes the drill bit 362 in a drill bit housing 363 and a mounting plate 364. The mounting plate 364 includes a set of basket wire apertures 366 providing for passage of the number of wires 308a-308d used in the lithotripsy basket 308 (such as, for example, four apertures for the illustrated 4-wire basket or six apertures for a 6-wire basket). FIG. 5A shows the same first drill head embodiment in a partial sectional-view illustration of the distal end of the outer sheath 306 in magnified detail with the drill head 360 mounted thereto. The mounting plate 364 is attached to the distal end of the outer sheath 306 (e.g., by a weld or strong adhesive), and includes a central aperture through which the drill bit housing 363 is mounted (e.g., by press-fit or other secure mounting means known in the art and configured to prevent proximal migration of the drill bit housing).

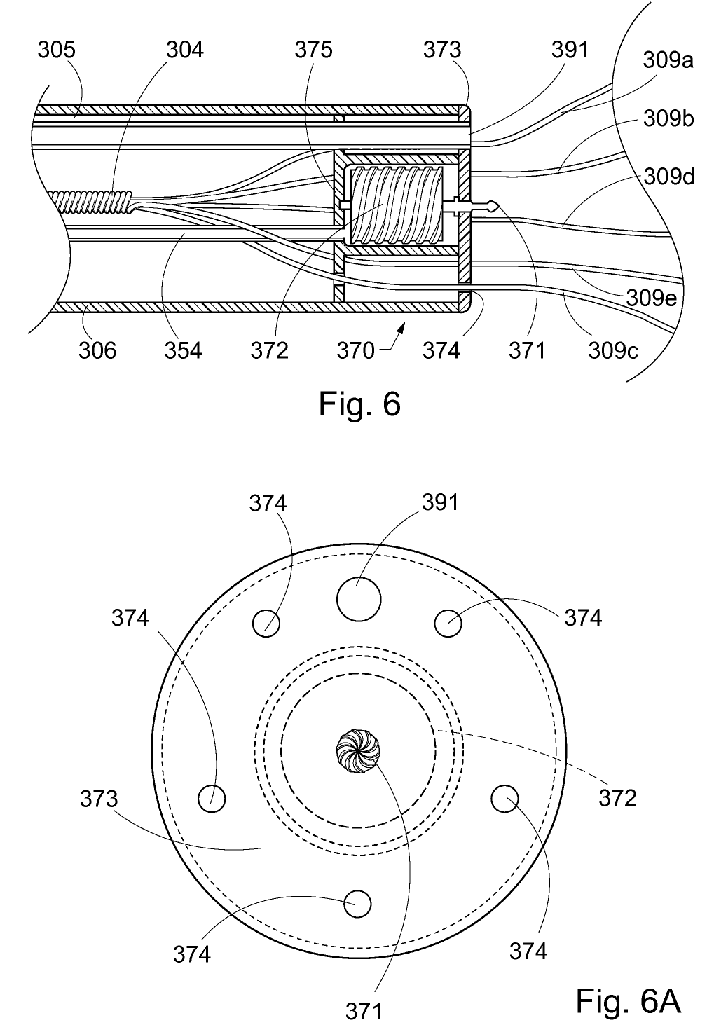

FIGS. 5-5A also illustrate a wire guide structure 379, which has a wire guide lumen extending lengthwise therethrough. Those of skill in the art will appreciate that the wire guide structure 379 is configured so that it may provide a short-wire-guided (also known as rapid exchange) functionality for directing the lithotriptor 300 along a wire guide. Those of skill in the art will also appreciate that a lumen (shown in FIG. 6 as a wire guide lumen 391) extending through the outer sheath 306 may be utilized alone to provide for “long wire” guidance capacity, or together with the wire guide structure such as, for example, a wire guide structure 379 to provide a convertible wire guide capacity (i.e., allowing for “short wire” or “long wire” use).

FIG. 6 depicts a partial sectional-view of second embodiment of a drill head 370. The drill head 370 includes a drill bit 371 in a drill bit housing 372 and a cup-shaped mounting bracket 373. The mounting bracket 373 includes a set of basket wire apertures 374 providing for passage of the number of wires 309a-309e used in the lithotripsy basket 308 (such as, for example, four apertures for a 4-wire basket or five apertures for a 5-wire basket). The mounting bracket 373 is attached about the distal end of the outer sheath 306 (e.g., by a weld, strong adhesive, crimp fit), and includes a central aperture through which the drill bit housing 372 is mounted. The inner diameter of the mounting bracket 373 is approximately the same as the outer diameter of the distal end of the outer sheath 306 (an end portion of which may be indented slightly as illustrated, or which may have the same outer diameter as a major length of the outer sheath 306). A securement plate 375 having a diameter approximately the same as the outer diameter of the distal end of the outer sheath 306 is mounted flush to that distal end and the drill bit housing 372 is secured between that plate 375 and the mounting bracket 373. The plate 375 includes apertures for passage of the basket wires 308a-308dand a wire guide lumen aperture 393, as well as for a fluid line 354 or an electronic communication line 384. A distal end view of the plate 375 is provided in FIG. 6A to more clearly illustrate the placement of the basket wire apertures 374, wire guide aperture 393, and the drill bit 371 in its housing 372.

Those of skill in the art will appreciate that, although the above-described embodiments have a drill assembly attached generally fixedly near the distal lithotriptor end, embodiments wherein the drill assembly or one or more parts thereof are movable (for example, retractable and/or extendable, or able to be angled) are within the scope of the present invention and may present advantages in introducing the device and/or contacting a stone with the drill.

A method of use is described with reference to FIGS. 7A-7E, which illustrate the method using the embodiment shown in FIGS. 3 and 5A, showing only external views (internal components are designated with reference to FIGS. 3 and 5A). Using a standard procedure such as ERCP, the lithotriptor device 300 is directed to a location adjacent a stone 311 to be extracted, as shown in FIG. 7A. Next, as shown in FIG. 7B, the basket 308 is deployed and opened by advancing the drive wire 304 distally through the outer sheath 306 such that the basket wires 308a-308d are advanced through the basket wire apertures 366 and the basket 308 is opened. Then, as depicted in FIG. 7C, the lithotriptor 300 and/or drive wire 304 are manipulated to capture the stone 311 in the basket 308. Next, the basket 308 is drawn compressingly around the stone 311. If the stone 311 is small enough to be withdrawn from its location intact, that action may be executed. If not, then—with the basket 308 drawn around the stone 311 in a manner that captures the stone against the drill bit 362—the drill mechanism 360 may be actuated to rotate the drill bit 362 bitingly against the stone 311 as illustrated in FIG. 7D. During this step, the handle 302 may be manipulated to move the basket 308 and the stone 311 to change position, angle, and force between the stone and the drill bit 362. Actuation of the drill mechanism 360 may be controlled by the actuation switch 352. (Those of skill in the art will appreciate that the actuation switch controls fluid flow in a fluid-driven-turbine drill embodiment, and controls an electrical signal in an electronically-driven drill embodiment, as well as that other currently known or future-developed drill control embodiments are useful within the scope of the present invention). The mechanical disruptive action of the drill bit 362 as well as an accompanying vibration of the drilling upon the stone 311, combined with an increased compressive pressure by actuation of the lithotripsy basket 308, will enhance the likelihood of fragmenting the stone 311 into two or more fragments as shown in FIG. 7E. Thereafter, the fragments of the stone 311 may be captured and extracted, or allowed to pass without assistance.

It is therefore intended that the foregoing detailed description be regarded as illustrative rather than limiting. It should be understood that the following claims, including all equivalents, are intended to define the spirit and scope of this invention.

I claim:

1. A lithotriptor device comprising:
a proximal handle;
an elongate shaft extending distally from the handle with a lumen extending through a major length of the elongate shaft;
a wire basket distally attached to a drive wire, the drive wire extending through the lumen of the elongate shaft and operatively connected to the handle; and
a drill mechanism assembly comprising a drill bit and disposed near the distal end of the elongate shaft.

2. The lithotriptor device of claim 1, wherein the drill mechanism assembly comprises a fluid-driven turbine.

3. The lithotriptor device of claim 1, wherein the drill mechanism assembly comprises an electronically-driven rotor.

4. The lithotriptor device of claim 1, wherein the drill mechanism assembly comprises a mounting plate securing the drill mechanism assembly to the elongate shaft.

5. The lithotriptor device of claim 1, further comprising a wire guide lumen structure.

6. The lithotriptor device of claim 5, wherein the wire guide lumen structure is mounted outside the lumen of the elongate shaft.

7. The lithotriptor device of claim 1, wherein the handle comprises a switch that is controllingly connected with the drill mechanism.

8. The lithotriptor device of claim 7, wherein the control connection between the switch and the drill mechanism comprises a selected one of a fluid flow passage or an electronic communication connection.

9. The lithotriptor device of claim 7, wherein the switch is selected from a dual-state (on/off) switch and a rheostat switch.

10. The lithotriptor device of claim 1, wherein the elongate shaft comprises a generally cylindrical metal coil.

11. The lithotriptor device of claim 1, wherein the basket and the drill mechanism are configured and disposed such that an object drawn proximally in the basket will contact the drill bit of the drill mechanism

12. A method for crushing an object, said method comprising the steps of:
providing a medical lithotriptor device comprising:
a proximal handle;
an elongate shaft extending distally from the handle with a lumen extending through a major length of the elongate shaft;
a basket distally attached to a drive wire, the drive wire extending through the lumen of the elongate shaft and operatively connected to the handle; and
a drill mechanism assembly comprising a drill bit and disposed near the distal end of the elongate shaft.
engaging the basket around an object; and
actuating the handle such that the drive wire is drawn proximally into the elongate shaft and the basket is drawn tightly around the object.

13. The method of claim 12, further comprising a step of actuating the drill mechanism such that the drill bit contacts the object.

14. The method of claim 13, further comprising a step of actuating the handle such that a tension of the basket around the object is reduced.

15. The method of claim 12, wherein the drill mechanism is powered by a selected one of a fluid-driven turbine or an electric motor.

16. The method of claim 12, further comprising a step of breaking the object into two or more fragments.

17. The method of claim 16, further comprising a step of using the basket to move at least one of the two or more fragments from a first location to a second location.

18. The method of claim 12, wherein the lithotriptor device further comprises a wire guide lumen structure.

19. The method of claim 18, wherein the wire guide lumen structure is mounted outside the lumen of the elongate shaft.

20. A lithotriptor device comprising:
a proximal handle;
an elongate shaft extending distally from the handle with a lumen extending through a major length of the elongate shaft;
a wire basket distally attached to a drive wire, the drive wire extending through the lumen of the elongate shaft and operatively connected to the handle; and
a fluid turbine-driven drill means comprising a drill bit, said drill means being disposed such that the drill bit projects distally from the elongate shaft.
